(19) United States
(12) Patent Application Publication
(10) Pub. No.: US 2022/0323547 A1
(43) Pub. Date: Oct. 13, 2022

(54) Amelioration and Treatment of Opioid Addiction

(71) Applicant: Therapeutic Solutions International, Inc., Oceanside, CA (US)

(72) Inventors: Thomas E. Ichim, Oceanside, CA (US); Timothy G. Dixon, Oceanside, CA (US); James Veltmeyer, Oceanside, CA (US); Kalina O'Connor, Oceanside, CA (US)

(73) Assignee: Therapeutic Solutions International, Inc., Oceanside, CA (US)

(21) Appl. No.: 17/720,240

(22) Filed: Apr. 13, 2022

Related U.S. Application Data
(60) Provisional application No. 63/174,291, filed on Apr. 13, 2021.

(51) Int. Cl. A61K 38/24; A61K 35/51; A61K 38/095; A61K 31/09; A61P 25/28

(52) U.S. Cl. CPC A61K 38/24; A61K 35/51; A61K 38/095; A61K 31/09; A61P 25/28

(57) ABSTRACT

Disclosed are compositions of matter, protocols and treatment means for reducing and/or preventing opioid addiction. In one embodiment the invention teaches intranasal administration of umbilical cord blood plasma, or extracts thereof, together with pterostilbene or pterostilbene containing nanoparticles, and/or oxytocin, and/or human chorionic gonadotropin.

### CROSS-REFERENCE TO RELATED APPLICATIONS

This application claims priority to U.S. Provisional Application Ser. No. 63/174,291, filed on Apr. 13, 2021, entitled “Amelioration and Treatment of Opioid Addiction”, which is incorporated herein by reference in its entirety.

### FIELD OF THE INVENTION

The invention belongs to the area of ameliorating and treating opioid addictions by administering umbilical cord blood plasma along with other active agents.

### BACKGROUND OF THE INVENTION

According to various key opinion leaders, it is believed that somewhere between 26.4 million to 36 million people abuse opioids such as heroin, morphine, and other prescription pain relievers internationally. Unfortunately, there is evidence that the opioid misuse is increasing. For example, the number of unintentional overdose deaths from opioid prescription analgesics has soared in the United States, more than quadrupling since 1999. It is estimated that in 2017 approximately 70,000 Americans died of opioid overdose¹. Current treatments for opioid addiction includes opioid antagonists such as naltrexone. This approach has been shown to curb drug craving and prevent relapse. Unfortunately, naltrexone possesses severe side effects such as depression, dysphoria, pulmonary edema and cardiac arrhythmias in certain cases.

For other drugs associated with various types of addictions, including cocaine and nicotine, immunological means of addressing these addictions have been attempted. Unfortunately means of inducing strong immune response to these drugs and/or receptors of these drugs are not currently available. Furthermore, there is always the possibility of inducing autoimmunity in central nervous system tissues. The current invention provides the new, useful and non-obvious utilization of microbiome manipulation using administration of probiotics and/or enzymes in order to treated addictions.

### SUMMARY

Preferred methods involve treating opioid addiction comprising administration of a composition containing cord blood, and/or oxytocin, and/or pterostilbene, and/or human chorionic gonadotropin.

Preferred methods include embodiments wherein said composition is administered intranasally.

Preferred methods include embodiments wherein said composition is administered for the purpose of reducing TLR4 expression in the brain.

Preferred methods include embodiments wherein said composition is administered for the purpose of reducing TNF-alpha expression in the brain.

Preferred methods include embodiments wherein said composition is administered for the purpose of reducing IL-1 beta expression in the brain.

Preferred methods include embodiments wherein said composition is administered for the purpose of reducing IL-6 beta expression in the brain.

Preferred methods include embodiments wherein said composition is administered for the purpose of reducing IL-11 beta expression in the brain.

Preferred methods include embodiments wherein said composition is administered for the purpose of reducing IL-12 beta expression in the brain.

Preferred methods include embodiments wherein said composition is administered for the purpose of reducing IL-15 beta expression in the brain.

Preferred methods include embodiments wherein said composition is administered for the purpose of reducing IL-17 beta expression in the brain.

Preferred methods include embodiments wherein said composition is administered for the purpose of reducing IL-18 beta expression in the brain.

Preferred methods include embodiments wherein said composition is administered for the purpose of increasing BDNF expression in the brain.

Preferred methods include embodiments wherein said composition is administered for the purpose of increasing NGF expression in the brain.

Preferred methods include embodiments wherein said composition is administered for the purpose of increasing IL-4 expression in the brain.

Preferred methods include embodiments wherein said composition is administered for the purpose of increasing IL-10 expression in the brain.

Preferred methods include embodiments wherein said composition is administered for the purpose of increasing IL-13 expression in the brain.

Preferred methods include embodiments wherein said composition is administered for the purpose of increasing neurogenesis.

Preferred methods include embodiments wherein said neurogenesis occurs in the dentate gyrus.

Preferred methods include embodiments wherein said neurogenesis occurs in the subventricular zone.

Preferred methods herein include measuring levels of the cytokines listed above, whether before and/or after administration to ascertain if they were in reduced or increased.

### BRIEF DESCRIPTION OF THE DRAWINGS

FIG. 1 is a line graph comparing the effects of cord blood plasma and the combination of cord blood plasma with pterostilbene on TLR4 expression.

### DESCRIPTION OF THE INVENTION

“Opioid addiction” or “Opioid Use Disorder” refers to a condition characterized by the harmful consequences of repeated opioid use, a pattern of compulsive opioid use, and sometimes physiological dependence on opioid including tolerance and/or symptoms of withdrawal. In the practice of the invention, opioid addiction is associated with enhanced inflammatory signaling [2-4].

“Drug withdrawal” refers to a group of symptoms that occur upon the abrupt discontinuation or sudden decrease in intake of medications or recreational drugs. Consequently, “opioid withdrawal” refers to the group of symptoms that occur upon the dramatic reduction, abrupt discontinuation or decrease in intake of opioids or opiates. Withdrawal symptoms may also start between doses. Withdrawal symptoms from opioids include but are not limited to anxiety, depression, sweating, vomiting, and diarrhea, muscle cramping, agitation, insomnia, yawning dilated pupils, goose bumps, abdominal cramping, runny nose and increased tearing, for example.

The term “substantially the same” or “not significantly different” refers to a level of expression that is not significantly different than what it is compared to. Alternatively, or in conjunction, the term substantially the same refers to a level of expression that is less than 2, 1.5, or 1.25 fold different than the expression or activity level it is compared to.

A “subject,” “individual” or “patient” is used interchangeably herein and refers to a vertebrate, for example a primate, a mammal or a human. Mammals include, but are not limited to equines, canines, bovines, ovines, murines, rats, simians, humans, farm animals, sport animals and pets. Also intended to be included as a subject are any subjects involved in clinical research trials not showing any clinical sign of disease, or subjects involved in epidemiological studies, or subjects used as controls.

“Diagnosis” may refer to the process of attempting to determine or identify a possible disease or disorder, or to the opinion reached by this process. From the point of view of statistics the diagnostic procedure may involve classification tests.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some embodiments it is contemplated that a numerical value discussed herein may be used with the term “about” or “approximately.” The term “about” or “around” is also used to indicate that a value includes the standard deviation of error for the device or method being employed to determine the value.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. “Consisting essentially of” in the context of pharmaceutical compositions of the disclosure is intended to include all the recited active agents and excludes any additional non-recited active agents, but does not exclude other components of the composition that are not active ingredients. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention. It is contemplated that embodiments described in the context of the term “comprising” may also be implemented in to context of the term “consisting of” or “consisting essentially of.”

The terms “protein”, “polypeptide” and “peptide” are used interchangeably herein when referring to a gene product or functional protein.

The terms “ameliorating,” “inhibiting,” or “reducing,” or any variation of these terms, when used in the claims and/or the specification includes any measurable decrease or complete inhibition to achieve a desired result.

The term “inhibitor” refers to a therapeutic agent that indirectly or directly inhibits the activity or expression of a protein, process (e.g. metabolic process), or biochemical pathway

The term “umbilical cord blood containing compositions” refers to therapeutic compositions for the practice of the invention which contain umbilical cord and/or concentrated umbilical cord, and/or one or more antioxidants, and/or one or more NF-kappa B inhibitors, and/or one or more stimulators of neurogenesis, and/or one or more inhibitors of neuroinflammation. In one embodiment said umbilical cord blood containing composition is comprised of umbilical cord blood together with oxytocin.

The term “agonist” describes a moiety or agent that interacts with and activates a receptor such as a G-protein-coupled receptor, for instance an opioid receptor, and can thereby initiate a physiological or pharmacological response characteristic of that receptor.

As used herein, a “partial agonist” is moiety, or agent, that binds to and activates a given receptor, but have only partial efficacy at the receptor relative to a full agonist.

As used herein an “antagonist” describes a moiety that competitively binds to the receptor at the same site as an agonist, but does not activate the intracellular response initiated by the active form of the receptor and can thereby inhibit the intracellular responses by an agonist or partial agonist.

The term “pharmaceutical formulation” is intended to mean a composition or a mixture of compositions comprising at least one active ingredient; including but not limited to, salts, solvates, and hydrates of compounds described herein.

As used herein, “treating,” “treatment” or “therapy” is an approach for obtaining beneficial or desired clinical results. This includes the reduction or the alleviation of symptoms, the reduction or alleviation of pain, or the reduction in the frequency of withdrawal symptoms, and/or reduction in the occurrence of anxiety or depression and/or reduction in suicidal thinking. Furthermore, these terms are intended to encompass curing as well as ameliorating at least one symptom of the condition or disease. For example, in the case of opioid use disorders, a response to treatment includes the cessation in the use of opioids, or the cessation of at least one opioid withdrawal symptom.

The term “therapeutically effective amount” refers to an amount of cells that treats or inhibits addiction, or withdrawal symptoms in the subject. In some embodiments, the therapeutically effective amount inhibits at least or at most or exactly 100, 99, 98, 96, 94, 92, 90, 85, 80, 75, 70, 65, 60, 55, 50, 40, 30, 20, or 10%, or any derivable range therein, of a symptom expression.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, etc.

As used herein, “allogeneic” refers to tissues or cells from another body that in a natural setting are immunologically incompatible or capable of being immunologically incompatible, although from one or more individuals of the same species.

As used herein, the term “allotransplantation” refers to the transplantation of organs, tissues, and/or cells from a donor to a recipient, where the donor and recipient are different individuals, but of the same species. Cells or tissue transplanted by such procedures is referred to as an allograft or allotransplant.

As used herein, “autologous” refers to tissues or cells that are derived or transferred from the same individual's body (i.e., autologous blood donation; an autologous bone marrow transplant).

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells that lack protein activity with an expression vector that expresses the protein, a derivative thereof, or a portion thereof. In other cases, a fragment of a gene product (such as a protein) may be considered biologically active (or it may be referred to as functionally active) if it retains the activity of the full-length gene product, although it may be at a reduced but detectable level of the activity of the full-length gene product.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Furthermore, an embodiment discussed in the Examples may be applied in the context of any other embodiments discussed herein.

The use of the word “a” or “an” when used in conjunction with the term “comprising” in the claims and/or the specification may mean “one,” but it is also consistent with the meaning of “one or more,” “at least one,” and “one or more than one.”

In one embodiment of the invention, administration of umbilical cord blood is provided together with agents that inhibit inflammation. In one embodiment agents that inhibit inflammation include omega 3 fatty acids, which alone, as well as with microbiome manipulation, have been shown to possess some ability to reduce opioid addiction [5-13]. The patent provides means of synergistically upregulating the anti-addictive properties of omega 3 fatty acids and/or probiotic interventions by co-administration of umbilical cord blood and/or hCG and/or oxytocin.

One pain mediator that may be targeted together with administration of umbilical cord plasma containing compositions is interleukin 6. It is known that spinal IL-6 levels correlated directly with the mechanical allodynia intensity following nerve injury.

One study sought to determine whether it is possible to attenuate mechanical allodynia and/or alter spinal glial activation resulting from peripheral nerve injury by specific manipulation of IL-6 with neutralizing antibodies or by global immune modulation utilizing immunogamma-globulin (IgG). Effects of peripheral administration of normal goat IgG and intrathecal (i.t.) administration of IL-6 neutralizing antibody, normal goat or normal rat IgG on mechanical allodynia associated with L5 spinal nerve transection were compared. Spinal glial activation was assessed at day 10 post surgery by immunohistochemistry. Low dose (0.01-0.001 microg) goat anti-rat IL-6 i.t. administration (P=0.025) significantly decreased allodynia and trended towards significance at the higher dose (0.08 microg to 0.008 microg, P=0.062). Low doses (0.01-0.001 microg) i.t. normal goat and rat IgG significantly attenuated mechanical allodynia, but not at higher doses (0.08-0.008 microg; P=0.001 for both goat and rat IgG). Peripherally administered normal goat IgG (30 or 100 mg/kg) did not attenuate mechanical allodynia. Thus in one embodiment of the invention, neutralization of IL-6 may be performed together with administration of probiotic, and/or probiotic/enzyme mixture, for the treatment of pain, and reduction of addiction associated with opioids [14]. The role of IL-6 in addiction is well known and one of skill in the art is referred to the following publications which are incorporated by reference [15-22].

In some studies a direct correlation has been shown between IL-6 levels and alcohol addiction. For example, Heberlein et al investigated the serum levels of IL-6 and TNF-α in 30 male alcohol-dependent patients during withdrawal (day 1, 7, and 14) and compared them with the levels obtained from 18 healthy male controls. IL-6 (day 1: T=2,593, p=0.013; day 7: T=2,315, p=0.037; day 14: T=1,650, p=0.112) serum levels were significantly increased at the beginning of alcohol withdrawal. TNF-α (T=3,202, p=0.03) serum levels were significantly elevated in the patients' group during the whole period of withdrawal. IL-6 serum levels decreased significantly during withdrawal (F=16.507, p<0.001), whereas TNF-α levels did not change significantly (day 1-14). IL-6 serum levels were directly associated with alcohol consumption (r=0.392, p=0.047) on day 1. Moreover, the IL-6 serum levels were associated with alcohol craving (PACS total score day 1: r=−0.417, p=0.022, the score of the obsessive subscale of the OCDS on day 14 [r=−0.549, p=0.022]), depression (r=−0.507, p=0.005), and trait anxiety (r=−0.674, p<0.001) on day 1. The authors found an association with the duration of active drinking following the last period of abstinence and the TNF-α serum levels (day 1:r=0.354, p=0.009; day 7: r=0.323, p=0.022; day 14: r=0.303, p=0.034) as well as an association with the severity of alcohol dependence measured by the SESA scale (r=0.454, p=0.015) [23].

In one embodiment of the invention, umbilical cord mixtures are administered together agents that prevent neuroinflammation, said agents include n-acetylcysteine [24],

In one embodiment of the invention, the invention teaches the use of probiotics and/or probiotic enzyme mixture to prevent brain atrophy associated with alcohol [25], or various drugs of addiction.

In one embodiment of the invention, the probiotic/prebiotic mixture is administered in order to enhance neurogenesis in patients who have suffered from brain damage as a result of opioid and/or other addictions. The study of adult neurogenesis has been previously described [26-29], and suppression of neurogenesis in alcohol, opioid, cocaine and methamphetamine addiction has been previously reported [30-40]. One of the aims of the current disclosure is to induce, and/or repair brain tissue that has been damage.

In one embodiment of the invention, stem cells are utilized to overcome drug induced neurotoxicity [41], and said probiotic/enzyme mixture is utilized to enhance efficacy of stem cells. Enhancement of efficacy comprises stimulation of growth factor production, inhibition of inflammation and triggering of mitogenesis of stem cells and existing endogenous regenerative cells.

In another embodiment of the invention, the umbilical cord plasma mixture is administered together with growth factors and/or hormones to stimulate regeneration of injured brain cells and decrease abnormalities that have been associated with suppression of neural pathways by addiction. Example include administration of growth hormone [42], agents which block adrenal hormones [43, 44], enhancement of serotonin [45-47], administration of FGF-2 [48, 49], antidepressants such as tranylcypromine, reboxetine, fluoxetine, haloperidol, tranylcypromine, reboxetine, fluoxetine, and haloperidol [50], electroconvulsive therapy [51], lithium [52], insulin like growth factor [53], inhibition of IL-6 [54], heparin binding epidermal growth factor like growth factor [55], VEGF [56], DHEA [57], BDNF [58], NMDA receptor antagonists [59], PGE2 [60], prolactin [61], the Chronic AMPA receptor potentiator (LY451646) [62], PACAP [63], lithium [64], transcranial magnetic field stimulation [65], olanzapine or fluoxetine [66], in order to overcome addiction associated hippocampal damage. It is known that the brain has the ability to produce new neural stem/progenitor cells (NSPCs) during adulthood. Hippocampus is one of the most plastic region of the brain, where granular cells in the dentate gyrus are born in adulthood. The precursors of these cells are placed in the subgranular zone (SGZ), the tissue between hilus and granule cell layer. One of the characteristic of adult-born neurons in the hippocampus is their specific electrophysiological capability for extreme changes required in early stages of maturation. This property is pivotal for formation of memories and further physiological action. The SGZ provides a proper niche for proliferation and differentiation of stem cells in dentate gyrus. In one embodiment, the administration of fibroblast cells, or activated fibroblast cells is performed in a manner capable of stimulating proliferation of SGZ cells so as to induce neuronal repair, and in some cases cause formation of memories that are not associated with the addictive memory.

In another embodiment, umbilical cord plasma is used to modulate the environment of SGZ cells. For example, umbilical cord plasma are administered to decrease inflammatory cytokine and inflammatory mediator production by astrocytes. It is known in the art that astrocytes as important cellular components of SGZ, play an active role in proliferation and neuronal fate commitment of NSPCs in part through release of molecular signals such as Wnt protein and sonic hedgehog (Shh). Thus in some embodiments of the disclosure, umbilical cord plasma are administered to overcome opioid associated alteration in fibroblast activity. For example, umbilical cord plasma have been shown to play essential roles in neural cell survival, immune responding, and modulation and metabolism of neurotransmitters. Therefore, each stimulant that can affect NSPCs or their niche in the hippocampus could make a vast modification in the memory and behavior. Bulk of studies have found the alterations in adult neurogenesis of hippocampus in neuropsycho-logical disorders such as depression, schizophrenia, bipolar disease, and addiction. Thus in one embodiment the disclosure encompasses the administration of fibroblasts to correct addiction associated alterations of hippocampal function.

In some embodiments of the disclosure, umbilical cord plasma that are therapeutically useful for the practice of the disclosure are cultured in a manner to produce proteins/peptides wherein said proteins/peptides include but are not limited to activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors alpha.sub.1beta.sub.1 and alpha. sub.2beta.sub.1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor alpha.sub.5beta.sub.1, Factor X, HB-EGF, HBNF, HGF, HUAF, heart derived inhibitor of vascular cell proliferation, Ill, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-beta, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alpha, transferrin, thrombospondin, urokinase, VEGF-A, VEGF-B, VEGF-C, VEGF-D, VEGF-E, VEGF, VEGF.sub.164, VEGI, EG-VEGF or any combination thereof.

### Example

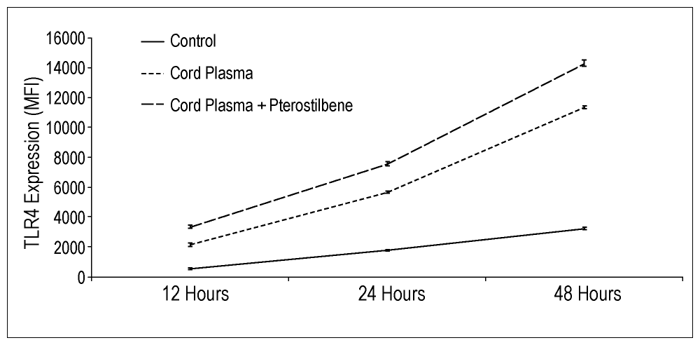

Neuroinflammation was induced by administration of LPS at 50 ng/mouse. Cord blood plasma and cord blood plasma with pterostilbene was administered intranasally. Assessment of TLR expression in brain homogenates were performed by flow cytometry. Results are shown in the line graph of FIG. 1.

### REFERENCES

1. Kimishima, A., et al., Cocaine Vaccine Development: Evaluation of Carrier and Adjuvant Combinations That Activate Multiple Toll-Like Receptors. Mol Pharm, 2016. 13(11): p. 3884-3890.
2. Lucerne, K. E. and D. D. Kiraly, The role of gut-immune-brain signaling in substance use disorders. Int Rev Neurobiol, 2021. 157: p. 311-370.
3. O'Sullivan, S. J. and J. S. Schwaber, Similarities in alcohol and opioid withdrawal syndromes suggest common negative reinforcement mechanisms involving the interoceptive antireward pathway. Neurosci Biobehav Rev, 2021. 125: p. 355-364.
4. Osmanlioglu, H. O., et al., Morphine Induces Apoptosis, Inflammation, and Mitochondrial Oxidative Stress via Activation of TRPM2 Channel and Nitric Oxide Signaling Pathways in the Hippocampus. Mol Neurobiol, 2020. 57(8): p. 3376-3389.
5. Hakimian, J. K., et al., Dietary Supplementation with Omega-3 Polyunsaturated Fatty Acids Reduces Opioid-Seeking Behaviors and Alters the Gut Microbiome. Nutrients, 2019. 11(8).
6. Maguire, D. and M. Groer, Neonatal abstinence syndrome and the gastrointestinal tract. Med Hypotheses, 2016. 97: p. 11-15.
7. Wang, F. and S. Roy, Gut Homeostasis, Microbial Dysbiosis, and Opioids. Toxicol Pathol, 2017. 45(1): p. 150-156.
8. Kang, M., et al., The effect of gut microbiome on tolerance to morphine mediated antinociception in mice. Sci Rep, 2017. 7: p. 42658.
9. Wang, F., et al., Morphine induces changes in the gut microbiome and metabolome in a morphine dependence model. Sci Rep, 2018. 8(1): p. 3596.
10. Meckel, K. R. and D. D. Kiraly, A potential role for the gut microbiome in substance use disorders. Psychopharmacology (Berl), 2019. 236(5): p. 1513-1530.
11. O'Sullivan, S. J., et al., Single-Cell Glia and Neuron Gene Expression in the Central Amygdala in Opioid Withdrawal Suggests Inflammation With Correlated Gut Dysbiosis. Front Neurosci, 2019. 13: p. 665.
12. Wang, F., et al., Opioid use potentiates the virulence of hospital-acquired infection, increases systemic bacterial dissemination and exacerbates gut dysbiosis in a murine model of Citrobacter rodentium infection. Gut Microbes, 2020. 11(2): p. 172-190.
13. Anderson, G., Pathoetiology and pathophysiology of borderline personality: Role of prenatal factors, gut microbiome, mu-and kappa-opioid receptors in amygdala-PFC interactions. Prog Neuropsychopharmacol Biol Psychiatry, 2020. 98: p. 109782.
14. Arruda, J. L., et al., Intrathecal anti-IL-6 antibody and IgG attenuates peripheral nerve injury-induced mechanical allodynia in the rat: possible immune modulation in neuropathic pain. Brain Res, 2000. 879(1-2): p. 216-25.
15. Zalcman, S., I. Savina, and R. A. Wise, Interleukin-6 increases sensitivity to the locomotor-stimulating effects of amphetamine in rats. Brain Res, 1999. 847(2): p. 276-83.
16. Nicolaou, C., et al., Serum cytokine concentrations in alcohol-dependent individuals without liver disease. Alcohol, 2004. 32(3): p. 243-7.
17. Ersche, K. D., et al., Aberrant disgust responses and immune reactivity in cocaine-dependent men. Biol Psychiatry, 2014. 75(2): p. 140-7.
18. Kane, C. J., et al., Effects of ethanol on immune response in the brain: region-specific changes in adolescent versus adult mice. Alcohol Clin Exp Res, 2014. 38(2): p. 384-91.
19. Fuster, D., et al., Inflammatory cytokines and mortality in a cohort of HIV-infected adults with alcohol problems. AIDS, 2014. 28(7): p. 1059-64.
20. Dahl, J., et al., The plasma levels of various cytokines are increased during ongoing depression and are reduced to normal levels after recovery. Psychoneuroendocrinology, 2014. 45: p. 77-86.
21. Neupane, S. P., et al., High frequency and intensity of drinking may attenuate increased inflammatory cytokine levels of major depression in alcohol-use disorders. CNS Neurosci Ther, 2014. 20(10): p. 898-904.
22. Chan, Y. Y., et al., Inflammatory response in heroin addicts undergoing methadone maintenance treatment. Psychiatry Res, 2015. 226(1): p. 230-4.
23. Heberlein, A., et al., TNF-alpha and IL-6 serum levels: neurobiological markers of alcohol consumption in alcohol-dependent patients? Alcohol, 2014. 48(7): p. 671-6.
24. Schneider, R., Jr., et al., N-acetylcysteine Prevents Alcohol Related Neuroinflammation in Rats. Neurochem Res, 2017. 42(8): p. 2135-2141.
25. Garcia-Valdecasas-Campelo, E., et al., Brain atrophy in alcoholics: relationship with alcohol intake; liver disease; nutritional status, and inflammation. Alcohol Alcohol, 2007. 42(6): p. 533-8.
26. Kaplan, M. S. and J. W. Hinds, Neurogenesis in the adult rat: electron microscopic analysis of light radioautographs. Science, 1977. 197(4308): p. 1092-4.
27. Lubbers, K., J. R. Wolff, and M. Frotscher, Neurogenesis of GABAergic neurons in the rat dentate gyrus: a combined autoradiographic and immunocytochemical study. Neurosci Lett, 1985. 62(3): p. 317-22.
28. Eriksson, P. S., et al., Neurogenesis in the adult human hippocampus. Nat Med, 1998. 4(11): p. 1313-7.
29. Roy, N. S., et al., In vitro neurogenesis by progenitor cells isolated from the adult human hippocampus. Nat Med, 2000. 6(3): p. 271-7.
30. Hauser, K. F., et al., Opioids intrinsically inhibit the genesis of mouse cerebellar granule neuron precursors in vitro: differential impact of mu and delta receptor activation on proliferation and neurite elongation. Eur J Neurosci, 2000. 12(4): p. 1281-93.
31. Eisch, A. J. and C. D. Mandyam, Drug dependence and addiction, II: Adult neurogenesis and drug abuse. Am J Psychiatry, 2004. 161(3): p. 426.
32. Fischer, S. J., et al., Morphine blood levels, dependence, and regulation of hippocampal subgranular zone proliferation rely on administration paradigm. Neuroscience, 2008. 151(4): p. 1217-24.
33. Cunha-Oliveira, T., A. C. Rego, and C. R. Oliveira, Cellular and molecular mechanisms involved in the neurotoxicity of opioid and psychostimulant drugs. Brain Res Rev, 2008. 58(1): p. 192-208.
34. Jin, J., et al., Interaction of the mu-opioid receptor with GPR177 (Wntless) inhibits Wnt secretion: potential implications for opioid dependence. BMC Neurosci, 2010. 11: p. 33.
35. Zheng, H., P. Y. Law, and H. H. Loh, Non-Coding RNAs Regulating Morphine Function: With Emphasis on the In vivo and In vitro Functions of miR-190. Front Genet, 2012. 3: p. 113
36. Hafizi, M., et al., Exploring the enkephalinergic differentiation potential in adult stem cells for cell therapy and drug screening implications. In Vitro Cell Dev Biol Anim, 2012. 48(9): p. 562-9.
37. Zheng, H., et al., NeuroD modulates opioid agonist-selective regulation of adult neurogenesis and contextual memory extinction. Neuropsychopharmacology, 2013. 38(5): p. 770-7.
38. Bernstein, H. G., et al., Increased densities of nitric oxide synthase expressing neurons in the temporal cortex and the hypothalamic paraventricular nucleus of polytoxicomanic heroin overdose victims: possible implications for heroin neurotoxicity. Acta Histochem, 2014. 116(1): p. 182-90.
39. Teuchert-Noodt, G., R. R. Dawirs, and K. Hildebrandt, Adult treatment with methamphetamine transiently decreases dentate granule cell proliferation in the gerbil hippocampus. J Neural Transm (Vienna), 2000. 107(2): p. 133-43.
40. Yamaguchi, M., et al., Repetitive cocaine administration decreases neurogenesis in adult rat hippocampus. Ann N Y Acad Sci, 2004. 1025: p. 351-62.
41. Kazma, M., et al., Survival, differentiation, and reversal of heroin neurobehavioral teratogenicity in mice by transplanted neural stem cells derived from embryonic stem cells. J Neurosci Res, 2010. 88(2): p. 315-23.
42. Rhodin, A., et al., Recombinant human growth hormone improves cognitive capacity in a pain patient exposed to chronic opioids. Acta Anaesthesiol Scand, 2014. 58(6): p. 759-65
43. Cameron, H. A. and E. Gould, Adult neurogenesis is regulated by adrenal steroids in the dentate gyrus. Neuroscience, 1994. 61(2): p. 203-9.
44. Montaron, M. F., et al., Adrenalectomy increases neurogenesis but not PSA-NCAM expression in aged dentate gyrus. Eur J Neurosci, 1999. 11(4): p. 1479-85.
45. Brezun, J. M. and A. Daszuta, Depletion in serotonin decreases neurogenesis in the dentate gyrus and the subventricular zone of adult rats. Neuroscience, 1999. 89(4): p. 999-1002.
46. Gould, E., Serotonin and hippocampal neurogenesis. Neuropsychopharmacology, 1999. 21(2 Suppl): p. 46S-51S.
47. Brezun, J. M. and A. Daszuta, Serotonin may stimulate granule cell proliferation in the adult hippocampus, as observed in rats grafted with foetal raphe neurons. Eur J Neurosci, 2000. 12(1): p. 391-6.
48. Wagner, J. P., I. B. Black, and E. DiCicco-Bloom, Stimulation of neonatal and adult brain neurogenesis by subcutaneous injection of basic fibroblast growth factor. J Neurosci, 1999. 19(14): p. 6006-16.
49. Palmer, T. D., et al., Fibroblast growth factor-2 activates a latent neurogenic program in neural stem cells from diverse regions of the adult CNS. J Neurosci, 1999. 19(19): p. 8487-97.
50. Malberg, J. E., et al., Chronic antidepressant treatment increases neurogenesis in adult rat hippocampus. J Neurosci, 2000. 20(24): p. 9104-10.
51. Madsen, T. M., et al., Increased neurogenesis in a model of electroconvulsive therapy. Biol Psychiatry, 2000. 47(12): p. 1043-9.
52. Chen, G., et al., Enhancement of hippocampal neurogenesis by lithium. J Neurochem, 2000. 75(4): p. 1729-34.
53. O'Kusky, J. R., P. Ye, and A. J. D'Ercole, Insulin-like growth factor-I promotes neurogenesis and synaptogenesis in the hippocampal dentate gyrus during postnatal development. J Neurosci, 2000. 20(22): p. 8435-42.
54. Vallieres, L., et al., Reduced hippocampal neurogenesis in adult transgenic mice with chronic astrocytic production of interleukin-6. J Neurosci, 2002. 22(2): p. 486-92.
55. Jin, K., et al., Heparin-binding epidermal growth factor-like growth factor: hypoxia-inducible expression in vitro and stimulation of neurogenesis in vitro and in vivo. J Neurosci, 2002. 22(13): p. 5365-73.
56. Jin, K., et al., Vascular endothelial growth factor (VEGF) stimulates neurogenesis in vitro and in vivo. Proc Natl Acad Sci USA, 2002. 99(18): p. 11946-50.
57. Karishma, K. K. and J. Herbert, Dehydroepiandrosterone (DHEA) stimulates neurogenesis in the hippocampus of the rat, promotes survival of newly formed neurons and prevents corticosterone-induced suppression. Eur J Neurosci, 2002. 16(3): p. 445-53.
58. Lee, J., W. Duan, and M. P. Mattson, Evidence that brain-derived neurotrophic factor is required for basal neurogenesis and mediates, in part, the enhancement of neurogenesis by dietary restriction in the hippocampus of adult mice. J Neurochem, 2002. 82(6): p. 1367-75.
59. Nacher, J., et al., NMDA receptor antagonist treatment increases the production of new neurons in the aged rat hippocampus. Neurobiol Aging, 2003. 24(2): p. 273-84.
60. Uchida, K., et al., Stimulatory effects of prostaglandin E2 on neurogenesis in the dentate gyrus of the adult rat. Zoolog Sci, 2002. 19(11): p. 1211-6.
61. Shingo, T., et al., Pregnancy-stimulated neurogenesis in the adult female forebrain mediated by prolactin. Science, 2003. 299(5603): p. 117-20.
62. Bai, F., M. Bergeron, and D. L. Nelson, Chronic AMPA receptor potentiator (LY 451646) treatment increases cell proliferation in adult rat hippocampus. Neuropharmacology, 2003. 44(8): p. 1013-21.
63. Mercer, A., et al., PACAP promotes neural stem cell proliferation in adult mouse brain. J Neurosci Res, 2004. 76(2): p. 205-15.
64. Kim, J. S., et al., Lithium selectively increases neuronal differentiation of hippocampal neural progenitor cells both in vitro and in vivo. J Neurochem, 2004. 89(2): p. 324-36.
65. Arias-Carrion, O., et al., Neurogenesis in the subventricular zone following transcranial magnetic field stimulation and nigrostriatal lesions. J Neurosci Res, 2004. 78(1): p. 16-28.
66. Kodama, M., T. Fujioka, and R. S. Duman, Chronic olanzapine or fluoxetine administration increases cell proliferation in hippocampus and prefrontal cortex of adult rat. Biol Psychiatry, 2004. 56(8): p. 570-80.

1. A method of treating opioid addiction comprising:
identifying a patient suffering from an opioid addiction;
providing a composition comprising cord blood, and an agent selected from the group consisting of: oxytocin, pterostilbene, and human chorionic gonadotropin; and
administering said composition to said patient in an amount sufficient to ameliorate or treat said opioid addiction.

2. The method of claim 2, wherein said composition is administered intranasally.

3. The method of claim 1, wherein said composition is administered for the purpose of reducing TLR4 expression in the brain.

4. The method of claim 1, wherein said composition is administered for the purpose of reducing TNF-alpha expression in the brain.

5. The method of claim 1, wherein said composition is administered for the purpose of reducing IL-1 beta expression in the brain.

6. The method of claim 1, wherein said composition is administered for the purpose of reducing IL-6 beta expression in the brain.

7. The method of claim 1, wherein said composition is administered for the purpose of reducing IL-11 beta expression in the brain.

8. The method of claim 1, wherein said composition is administered for the purpose of reducing IL-12 beta expression in the brain.

9. The method of claim 1, wherein said composition is administered for the purpose of reducing IL-15 beta expression in the brain.

10. The method of claim 1, wherein said composition is administered for the purpose of reducing IL-17 beta expression in the brain.

11. The method of claim 1, wherein said composition is administered for the purpose of reducing IL-18 beta expression in the brain.

12. The method of claim 1, wherein said composition is administered for the purpose of increasing BDNF expression in the brain.

13. The method of claim 1, wherein said composition is administered for the purpose of increasing NGF expression in the brain.

14. The method of claim 1, wherein said composition is administered for the purpose of increasing IL-4 expression in the brain.

15. The method of claim 1, wherein said composition is administered for the purpose of increasing IL-10 expression in the brain.

16. The method of claim 1, wherein said composition is administered for the purpose of increasing IL-13 expression in the brain.

17. The method of claim 1, wherein said composition is administered for the purpose of increasing neurogenesis.

18. The method of claim 17, wherein said neurogenesis occurs in the dentate gyrus.

19. The method of claim 17, wherein said neurogenesis occurs in the subventricular zone.
